(19) United States
(12) Patent Application Publication
(10) Pub. No.: US 2022/0167808 A1
(43) Pub. Date: Jun. 2, 2022

(54) AUTONOMOUS CLEANER AND CLEANING SYSTEM

(71) Applicant: Panasonic Intellectual Property Management Co., Ltd., Osaka (JP)

(72) Inventors: Tomoaki INOUE, Kyoto (JP); Koji ASAI, Osaka (JP); Yuko TSUSAKA, Kyoto (JP)

(21) Appl. No.: 17/406,004

(22) Filed: Aug. 18, 2021

(30) Foreign Application Priority Data
Nov. 27, 2020 (JP) 2020-196946

(51) Int. Cl. A47L 7/00; A47L 9/00; A47L 9/28; A61L 2/22

(52) U.S. Cl. CPC A47L 7/0061; A47L 9/009; A47L 9/2852; A47L 9/2805; A47L 9/2894; A61L 2202/17; A47L 9/2847; A61L 2/22; A47L 2201/04; A47L 2201/06; A47L 9/2857

(57) ABSTRACT

An autonomous cleaner includes: a main body including a housing, a drive wheel (wheel) attached to the housing, and a drive unit that drives the drive wheel; a map storage unit that stores map information for the main body to travel; a determination unit that determines whether the main body approaches or comes into contact with a person infected with at least one of a virus and a bacterium; and a map information corrector that reflects, in the map information stored in the map storage unit, contact information on approach or contact of the main body with the infected person determined by the determination unit.

### BACKGROUND

### 1. Technical Field

The present disclosure relates to an autonomous cleaner that autonomously travels and cleans a predetermined space, and a cleaning system.

### 2. Description of the Related Art

For example, WO 2019/064862A (hereinafter, referred to as “Patent Literature 1”) discloses a collection device for collecting an object such as a virus or a bacterium from a floor surface as an example of an autonomous cleaner.

However, the collection device disclosed in Patent Literature 1 only collects a virus and a bacterium, and a countermeasure cannot be taken against an infected person (including a person who has had close contact with an infected person).

### SUMMARY

The present disclosure provides an autonomous cleaner and a cleaning system capable of coping with a person infected with a virus or a bacterium.

An autonomous cleaner according to one aspect of the present disclosure includes: a main body including a housing, a drive wheel attached to the housing, and a drive unit that drives the drive wheel; a map storage unit that stores map information for the main body to travel; a determination unit that determines whether or not the main body approaches or comes into contact with a person infected with at least one of a virus and a bacterium; and a map information corrector that reflects, in the map information stored in the map storage unit, contact information on approach or contact of the main body with the infected person determined by the determination unit.

In addition, a cleaning system according to one aspect of the present disclosure is a cleaning system including: an autonomous cleaner; and a determination device configured to freely communicate with the autonomous cleaner. The autonomous cleaner includes a main body having a housing, a drive wheel attached to the housing, and a drive unit that drives the drive wheel, a map storage unit that stores map information for the main body to travel, a communication unit that communicates with a determination device, and a map information corrector that corrects the map information stored in the map storage unit. The determination device determines whether or not the determination device approaches or comes into contact with a person infected with at least one of a virus and a bacterium. The map information corrector reflects, in the map information stored in the map storage unit, contact information on approach or contact of the determination device with the infected person, which is determined by the determination device.

According to the present disclosure, it is possible to provide an autonomous cleaner and a cleaning system capable of coping with a person infected with a virus or a bacterium.

### BRIEF DESCRIPTION OF THE DRAWINGS

FIG. 1 is a side view illustrating an appearance of an autonomous cleaner according to an exemplary embodiment;

FIG. 2 is a front view illustrating an appearance of the autonomous cleaner according to the exemplary embodiment;

FIG. 3 is a bottom view illustrating an appearance of the autonomous cleaner according to the exemplary embodiment;

FIG. 4 is a block diagram illustrating a characteristic functional configuration of the autonomous cleaner according to the exemplary embodiment;

FIG. 5 is a table illustrating an example of a travel pattern database held by the autonomous cleaner according to the exemplary embodiment;

FIG. 6 is a flowchart illustrating processing executed by the autonomous cleaner according to the exemplary embodiment;

FIG. 7 is a flowchart illustrating map information correction processing executed by the autonomous cleaner according to the exemplary embodiment;

FIG. 8 is an explanatory diagram illustrating an example of a process when the autonomous cleaner according to the exemplary embodiment cleans a predetermined space;

FIG. 9 is an explanatory diagram illustrating a map based on map information corrected by a map information corrector according to the exemplary embodiment;

FIG. 10 is an explanatory diagram illustrating an example of a cleaning plan in a countermeasure mode according to the exemplary embodiment;

FIG. 11 is an explanatory diagram illustrating an example of a map displayed on an input unit according to the exemplary embodiment after completion of cleaning in the countermeasure mode;

FIG. 12 is an explanatory diagram illustrating a case where an autonomous cleaner according to a modification transmits contact information to a communication terminal of another person; and

FIG. 13 is a block diagram illustrating a characteristic functional configuration of a cleaning system according to a modification.

### DETAILED DESCRIPTION

Hereinafter, exemplary embodiments of an autonomous cleaner or the like according to the present disclosure will be described in detail with reference to the drawings. Note that each of the exemplary embodiments described below illustrates a preferred specific example of the present disclosure. Therefore, numerical values, shapes, materials, components, arrangement and connection forms of the components, steps, orders of steps, etc., to be used in the following exemplary embodiments are illustrative and are not to limit the scope of the present disclosure.

Note that the attached drawings and the following description are provided for those skilled in the art to fully understand the present disclosure, and are not intended to limit the subject matter as described in the appended claims.

Further, each of the drawings is a schematic diagram, and is not necessarily strictly illustrated. Furthermore, in each of the drawings, substantially the same components are denoted by the same reference numerals, and redundant description may be omitted or simplified.

Furthermore, in the following exemplary embodiments, an expression using “substantially” such as a substantially triangular shape is used. For example, a substantially cylindrical shape means not only a completely cylindrical shape but also a substantially cylindrical shape. That is, for example, a substantially cylindrical shape also means that a cylinder including some irregularities on a surface is included. The same applies to other expressions using “substantially”.

Further, in the following exemplary embodiments, a case where an autonomous cleaner that performs cleaning by traveling on a floor of a predetermined space is viewed from vertically above may be referred to as a top view, and a case where the autonomous cleaner is viewed from vertically below may be referred to as a bottom view.

### Exemplary Embodiment

[Configuration]

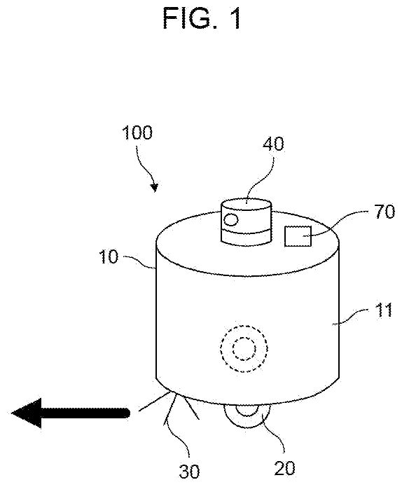

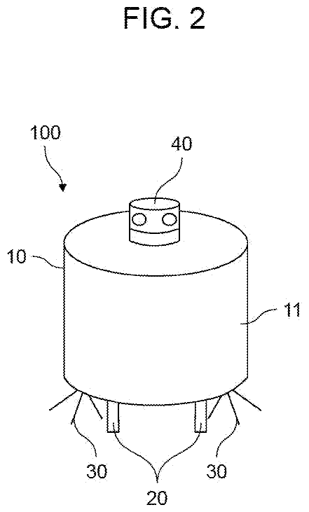

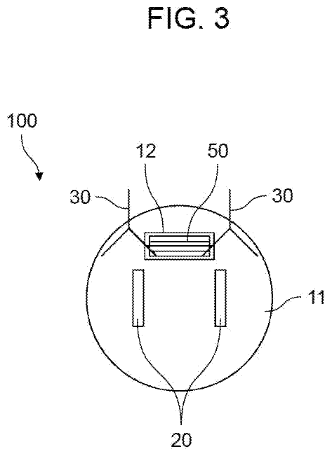

First, a configuration of autonomous cleaner 100 according to an exemplary embodiment will be described. FIG. 1 is a side view illustrating an appearance of autonomous cleaner 100 according to the exemplary embodiment. FIG. 2 is a front view illustrating an appearance of autonomous cleaner 100 according to the exemplary embodiment. FIG. 3 is a bottom view illustrating an appearance of autonomous cleaner 100 according to the exemplary embodiment.

Autonomous cleaner 100 is an autonomous cleaner that autonomously travels and cleans a predetermined space. First, autonomous cleaner 100 generates map information (data) indicating a map in a predetermined space by traveling around on a floor surface in the predetermined space.

Next, based on the generated map information, autonomous cleaner 100 calculates a travel route along which autonomous cleaner 100 travels when cleaning the predetermined space. Next, autonomous cleaner 100 travels in the predetermined space along the calculated travel route and performs cleaning.

Autonomous cleaner 100 autonomously determines whether to avoid an object (obstacle) present on a floor through observation using a sensor such as a cliff sensor. When an obstacle is present, autonomous cleaner 100 leaves the calculated travel route and travels and performs cleaning while avoiding the obstacle.

Autonomous cleaner 100 generates the map information of the predetermined space to be cleaned and estimates a self-position of autonomous cleaner 100 on the map indicated by the generated map information by simultaneous localization and mapping (SLAM), for example.

Autonomous cleaner 100 includes, for example, main body 10, two wheels 20, two side brushes 30, laser distance meter 40, main brush 50, and input unit 70.

Main body 10 accommodates components included in autonomous cleaner 100, and has cylindrical housing 11. Note that a shape of main body 10 in top view is not particularly limited. The shape of main body 10 in top view may be, for example, a substantially rectangular shape or a substantially triangular shape. As illustrated in FIG. 3, main body 10 has suction port 12 on a lower surface.

Two wheels 20 are drive wheels for causing autonomous cleaner 100 to travel, and are rotatably provided on the lower surface of main body 10.

Side brushes 30 are brushes that are provided on the lower surface of main body 10 and clean the floor surface of the predetermined space. In the present exemplary embodiment, autonomous cleaner 100 includes two side brushes 30. The number of side brushes 30 included in autonomous cleaner 100 may be one or three or more, and is not particularly limited.

Laser distance meter 40 is a sensor for measuring a distance between autonomous cleaner 100 and an object, a wall surface, or the like in the predetermined space. Laser distance meter 40 is, for example, a so-called light detection and ranging (LIDAR). Laser distance meter 40 is provided, for example, on an upper portion of main body 10.

Main brush 50 is disposed at suction port 12, and rotates to cause suction port 12 to suck dust on the floor surface.

Input unit 70 is disposed on an upper surface of main body 10 and behind laser distance meter 40. Input unit 70 is a portion that receives various instructions by being operated by a user. Specifically, input unit 70 is a touch panel. Therefore, input unit 70 also functions as a display unit that displays various types of information. Note that input unit 70 and the display unit may be separate bodies. Furthermore, input unit 70 may be a communication terminal such as a smartphone or a tablet terminal that can freely communicate with main body 10. In this case, main body 10 may be provided with an instrument to which the communication terminal is attachable.

The various instructions received by input unit 70 include a normal mode and a countermeasure mode. The countermeasure mode is a mode in which autonomous cleaner 100 takes a countermeasure against an infected person region where an infected person is present as described later. The normal mode is a mode in which so-called normal cleaning is executed without performing the countermeasure.

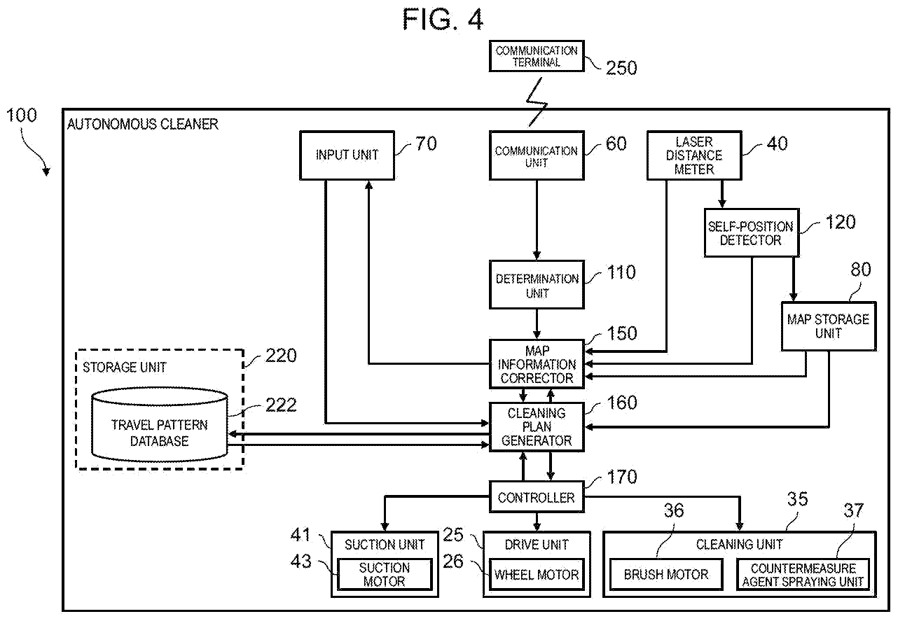

FIG. 4 is a block diagram illustrating a characteristic functional configuration of the autonomous cleaner according to the exemplary embodiment. As illustrated in FIG. 4, autonomous cleaner 100 includes laser distance meter 40, input unit 70, map storage unit 80, storage unit 220, communication unit 60, determination unit 110, self-position detector 120, map information corrector 150, cleaning plan generator 160, controller 170, suction unit 41, drive unit 25, and cleaning unit 35.

Map storage unit 80 stores map information generated by self-traveling of autonomous cleaner 100. The map information may be acquired from an external device. The map information includes a room layout, an obstacle, and the like in the predetermined space.

Communication unit 60 is a portion that communicates with external communication terminal 250. Specifically, communication unit 60 is a wireless interface that communicates with communication terminal 250 such as a smartphone or a tablet terminal owned by another person by a proximity communication function (Bluetooth). Here, it is assumed that communication terminal 250 has introduced an application, such as COVID-19 contact-confirming application (COCOA), that records whether communication terminal 250 has approached a person infected with at least one of a bacterium and a virus, and transmits content of the record. In addition, the application has a function of transmitting infected person information when a user is infected. The infected person information is information indicating that the user is an infected person. Note that the infected person may include a person who has had close contact with an infected person, and in this case, information indicating that the user is a person who has had close contact with an infected person is also included in the infected person information.

When communication unit 60 and communication terminal 250 enter a mutual communication range, communication unit 60 communicates with communication terminal 250 and acquires the infected person information transmitted from communication terminal 250. At this time, communication unit 60 also acquires signal strength during communication.

Determination unit 110 determines whether main body 10 approaches or comes into contact with the infected person based on the infected person information and the signal strength received by communication unit 60. Specifically, upon receiving the infected person information, from the signal strength received at the same time, determination unit 110 calculates a distance between main body 10 and communication terminal 250 during communication. When the distance calculated here is less than a predetermined value, determination unit 110 determines that main body 10 approaches or comes into contact with an infected person. On the other hand, in a case where the distance is the calculated predetermined value or more, determination unit 110 determines that main body 10 does not approach or come in contact with an infected person. The predetermined value is preferably in a range in which droplet infection of a target harmful substance is likely to occur. For example, in the case of COVID-19, it is said that droplet infection is less likely to occur when the distance between an infected person and a non-infected person is 2 m or more. Therefore, when the target harmful substance is COVID-19, the predetermined value may be set to 2 m. Determination unit 110 creates, as determination time information, a history of time when determination unit 110 determines that main body 10 approaches or comes in contact with an infected person, and outputs the determination time information to map information corrector 150.

Self-position detector 120 detects a position of autonomous cleaner 100 in a predetermined space. For example, self-position detector 120 calculates coordinates of autonomous cleaner 100 on the map indicated by the map information, based on a distance from an object including an obstacle, a wall, or the like, which is located around autonomous cleaner 100, and input from laser distance meter 40, and the map information in map storage unit 80. The self-position detector 120 outputs self-position information indicating the detected self-position to map information corrector 150 in association with the time of detection or the like.

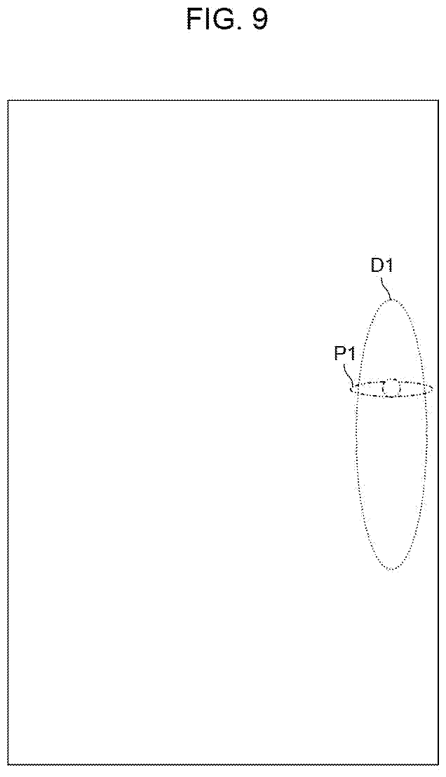

Map information corrector 150 reflects the contact information on approach or contact of main body 10 with an infected person, which is determined by determination unit 110, in the map information stored in map storage unit 80. Specifically, map information corrector 150 collates the determination time information acquired from determination unit 110 with the self-position information acquired from self-position detector 120 and the detection time, thereby associating the time when it is determined that main body 10 approaches or comes in contact with the infected person, with the self-position information, to create the contact information. With this contact information, the self-position of main body 10 at the time when main body 10 approaches or comes into contact with the infected person is specified. Map information corrector 150 reflects, in the map information in map storage unit 80, harmful concentrated region D1 (see FIG. 9) based on the contact information. Harmful concentrated region D1 is a region where an infected person has been present, and is a region where a harmful substance can be generated. Based on the self-position of main body 10 included in the contact information, map information corrector 150 reflects, in the map information, a range having a width of about a predetermined value as harmful concentrated region D1.

Storage unit 220 is a storage device that stores travel pattern database 222. Storage unit 220 is realized by, for example, a hard disk drive (HDD), a flash memory, or the like. Furthermore, storage unit 220 stores, for example, control programs executed by various processors such as controller 170.

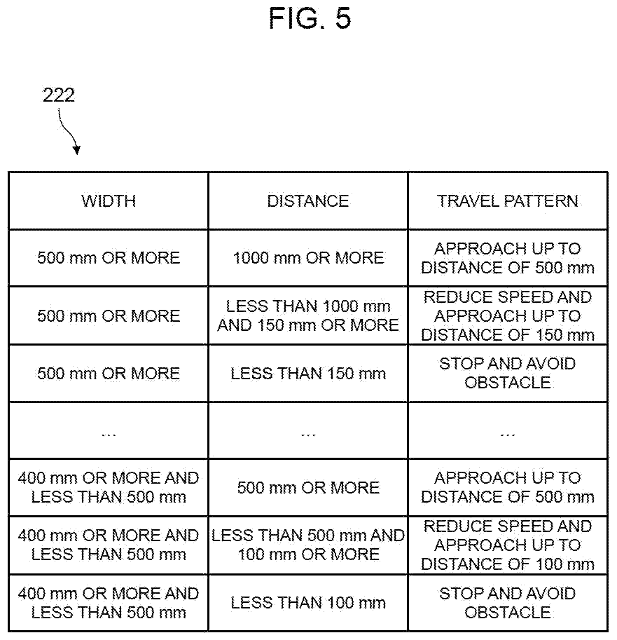

FIG. 5 is a table illustrating an example of travel pattern database 222 held by autonomous cleaner 100 according to the exemplary embodiment. In travel pattern database 222, a “width” is a width of an obstacle which main body 10 approaches, and a “distance” is a distance from main body 10 to the obstacle. Further, in travel pattern database 222, a “travel pattern” is an action of autonomous cleaner 100 toward the obstacle.

For example, when autonomous cleaner 100 approaches an obstacle having a width of 500 mm or less at a distance of 1000 mm or more, autonomous cleaner 100 thereafter performs an action of approaching up to 500 mm from the obstacle based on travel pattern database 222. Travel pattern database 222 is used when cleaning plan generator 160 generates a cleaning plan.

As illustrated in FIG. 4, cleaning plan generator 160 is a processor that generates an appropriate cleaning plan according to the normal mode or the countermeasure mode received by input unit 70. For example, cleaning plan generator 160 is a processor that generates a cleaning plan (plan information) indicating how autonomous cleaner 100 travels in a predetermined space for cleaning.

In the normal mode, based on the map information acquired from map storage unit 80, cleaning plan generator 160 generates a cleaning plan in which a travel route of autonomous cleaner 100, specifically, a travel method is determined which is a method of controlling drive unit 25 such as rotation speed of wheel motor 26 and a direction of wheels 20.

In addition, cleaning plan generator 160 generates a cleaning plan indicating a cleaning method including a method of controlling suction unit 41 (for example, a suction force, more specifically, rotation speed of suction motor 43), the method of controlling drive unit 25 such as the rotation speed of wheel motor 26 and the direction of wheels 20, a method of controlling cleaning unit 35 (for example, the number of rotations of brush motor 36), and the like. That is, in the normal mode, countermeasure agent spraying unit 37 is not driven, and a countermeasure agent is not sprayed.

On the other hand, in the countermeasure mode, based on the corrected map information acquired from map information corrector 150, cleaning plan generator 160 generates a cleaning plan in which the travel route of autonomous cleaner 100, specifically, the travel method which is the method of controlling drive unit 25 such as the rotation speed of wheel motor 26 and the direction of wheels 20 is determined. Specifically, in the countermeasure mode, a cleaning plan passing harmful concentrated region D1 is determined.

In addition, cleaning plan generator 160 generates a cleaning plan indicating a cleaning method including the method of controlling drive unit 25 such as the rotation speed of wheel motor 26 and the direction of wheels 20, a method of controlling cleaning unit 35 (for example, the number of times of spraying by countermeasure agent spraying unit 37), and the like. That is, in the countermeasure mode, the countermeasure agent is sprayed to harmful concentrated region D1. In the countermeasure mode, suction unit 41 and brush motor 36 may or may not be driven.

In this manner, cleaning plan generator 160 generates different cleaning plans for the normal mode and the countermeasure mode, and causes controller 170 to control autonomous cleaner 100, more specifically, suction unit 41, drive unit 25, and cleaning unit 35 based on the generated cleaning plans.

Based on the plan information generated by cleaning plan generator 160, controller 170 controls suction unit 41, drive unit 25, and cleaning unit 35 to cause autonomous cleaner 100 to autonomously travel in a predetermined space to perform cleaning.

Various processors such as determination unit 110, self-position detector 120, map information corrector 150, and controller 170 are implemented by, for example, a control program for executing the above-described processing, a central processing unit (CPU) that executes the control program, a random access memory (RAM), and a read only memory (ROM). Each of these processors may be realized by one or a plurality of CPUs.

Suction unit 41 is a mechanism for sucking dust on a floor surface of a predetermined space by sucking the floor surface. Suction unit 41 includes, for example, suction motor 43.

Suction motor 43 is connected to a fan, and sucks dust on a floor surface by rotating the fan.

Drive unit 25 is a mechanism for causing autonomous cleaner 100 to travel. Drive unit 25 includes, for example, wheel motor 26. Wheel motor 26 is connected to wheels 20 and is a motor for rotationally driving wheels 20.

Since rotation of two wheels 20 of drive unit 25 is independently controlled, autonomous cleaner 100 can perform free traveling such as going straight, moving backward, left rotation, and right rotation. Note that autonomous cleaner 100 may further include wheels (auxiliary wheels) which are not rotated by wheel motor 26.

Cleaning unit 35 is an example of a countermeasure executer that executes a countermeasure including at least one of reduction and prevention of a harmful substance by cleaning a floor surface. Cleaning unit 35 includes, for example, brush motor 36 and countermeasure agent spraying unit 37.

Brush motor 36 is a motor that is connected to a brush such as main brush 50 and drives (rotates) the brush such as main brush 50.

Countermeasure agent spraying unit 37 is a nozzle unit that sprays a countermeasure agent for inactivating a harmful substance. The countermeasure agent includes at least one of a sterilizing agent, a virus-Removing Agent, an Antibacterial Agent, and an Antiviral Agent.

### [Processing Procedure]

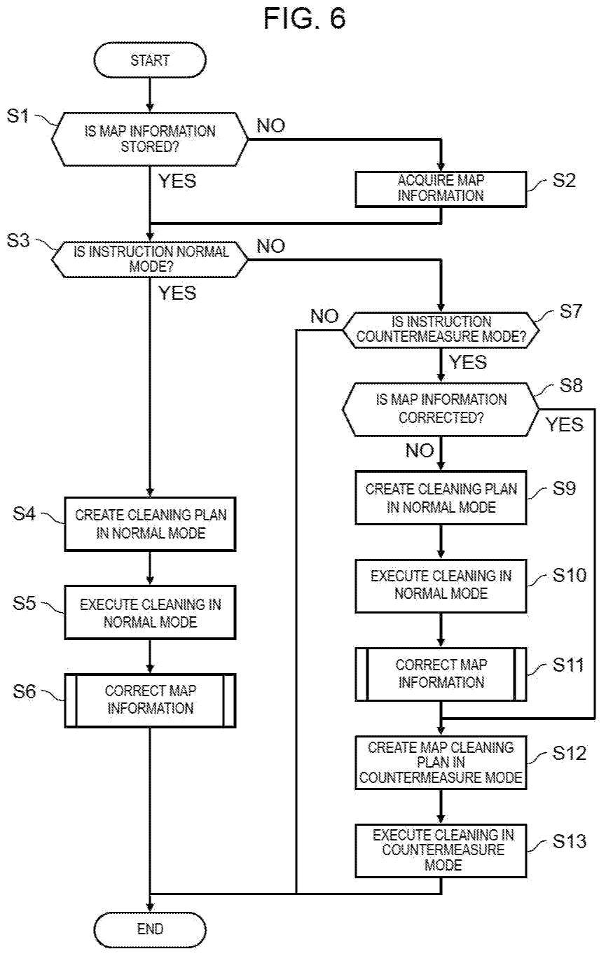

Next, an outline of a processing procedure of autonomous cleaner 100 will be described with reference to FIG. 6. FIG. 6 is a flowchart illustrating processing executed by autonomous cleaner 100 according to the exemplary embodiment.

In step S1, cleaning plan generator 160 determines whether map information is stored in map storage unit 80. In a case where the map information is not stored, the processing proceeds to step S2, and in a case where the map information is stored, the processing proceeds to step S3.

In step S2, cleaning plan generator 160 instructs controller 170 to acquire a map. By controlling drive unit 25 based on this instruction, controller 170 acquires and analyzes detection results of various sensors while causing main body 10 to travel in a predetermined space, and generates map information in the predetermined space. The generated map information is stored in map storage unit 80.

In step S3, cleaning plan generator 160 determines whether or not the instruction received by input unit 70 is the normal mode. In a case where the instruction is the normal mode, the processing proceeds to step S4. In a case where the instruction is not in the normal mode, the processing proceeds to step S7.

In step S4, cleaning plan generator 160 creates a cleaning plan corresponding to the normal mode.

In step S5, cleaning plan generator 160 executes cleaning in the normal mode based on the cleaning plan corresponding to the normal mode.

In step S6, map information corrector 150 reflects and corrects, in the map information stored in map storage unit 80, the harmful concentrated region determined by determination unit 110 during execution of cleaning in the normal mode.

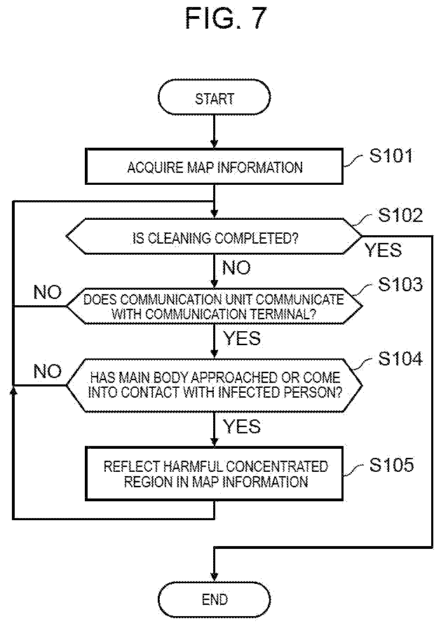

FIG. 7 is a flowchart illustrating map information correction processing executed by autonomous cleaner 100 according to the exemplary embodiment.

As illustrated in FIG. 7, in step S101, map information corrector 150 acquires map information from map storage unit 80.

In step S102, map information corrector 150 determines whether cleaning in the normal mode is completed, and ends the map information correction processing in a case where cleaning in the normal mode is completed.

In step S103, map information corrector 150 determines whether communication unit 60 communicates with communication terminal 250. In a case where communication unit 60 does not communicate with communication terminal 250, the processing proceeds to step S102. In a case where communication unit 60 communicates with communication terminal 250, the processing proceeds to step S104.

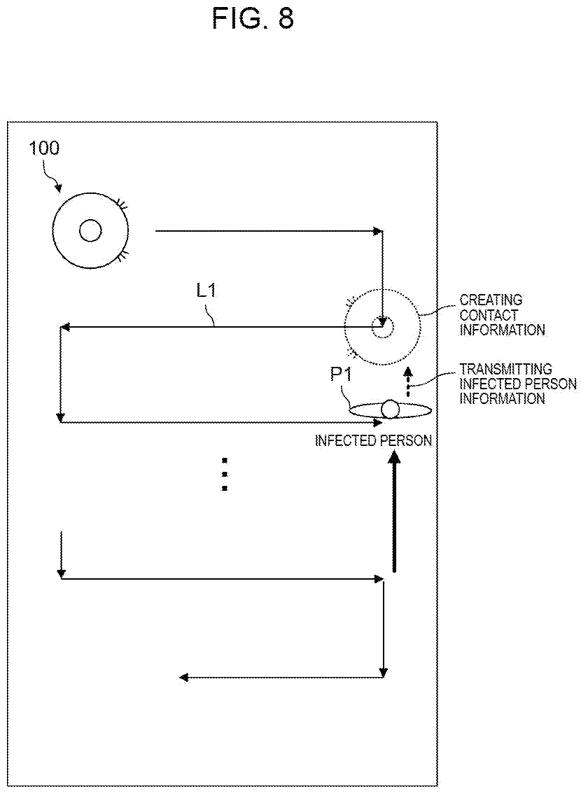

FIG. 8 is an explanatory diagram illustrating an example of a process when autonomous cleaner 100 according to the exemplary embodiment cleans a predetermined space. As illustrated in FIG. 8, when performing cleaning in the normal mode, autonomous cleaner 100 travels along travel route L1 corresponding to the cleaning plan. At the time of traveling, when person P1 is present near travel route L1, and communication terminal 250 possessed by person P1 and main body 10 of autonomous cleaner 100 enter the mutual communication range, communication unit 60 communicates with communication terminal 250 of person P1. For example, in a case where person P1 is an infected person, the infected person information is included in communication content and transmitted from communication terminal 250 possessed by person P1 to communication unit 60. Determination unit 110 creates the determination time information based on the communication content. When the communication content does not include the infected person information, determination unit 110 does not create the determination time information.

In step S104, map information corrector 150 determines whether main body 10 approaches or comes into contact with an infected person based on whether determination unit 110 has created the determination time information. In a case where map information corrector 150 determines that main body 10 has neither approached nor come into contact with the infected person, the processing proceeds to step S102, and in a case where map information corrector 150 determines that main body 10 has approached or has come into contact with the infected person, the processing proceeds to step S105,

In step S105, map information corrector 150 collates the determination time information acquired from determination unit 110 with the self-position information acquired from self-position detector 120 and the detection time, thereby associating the time when it is determined that main body 10 approaches or comes in contact with the infected person, with the self-position information, to create the contact information. Thereafter, map information corrector 150 reflects, in the map information stored in map storage unit 80, harmful concentrated region D1, based on the contact information and the processing proceeds to step S102.

FIG. 9 is an explanatory diagram illustrating a map based on the map information corrected by map information corrector 150 according to the exemplary embodiment. In FIG. 9, person P1 is indicated by a two-dot chain line for comparison with FIG. 8, but information on person P1 is not included in the actual map information. Map information corrector 150 causes input unit 70 to display a map based on the corrected map information. That is, the display unit including input unit 70 displays the map information in which the contact information is reflected. As a result, the user can visually recognize harmful concentrated region D1.

Returning to FIG. 6, in step S7, cleaning plan generator 160 determines whether or not the instruction received by input unit 70 is the countermeasure mode. In a case where the instruction is the countermeasure mode, the processing proceeds to step S8, and in a case where the instruction is not the countermeasure mode, the processing ends.

In step S8, cleaning plan generator 160 determines whether or not the map information has been corrected. In a case where the map information has been corrected, the processing proceeds to step S12, and in a case where the map information has not been corrected, the processing proceeds to step S9.

In step S9, cleaning plan generator 160 creates a cleaning plan in the normal mode in order to correct the map information. Note that, since this cleaning plan is for the purpose of correcting the map information, driving of cleaning unit 35 may not be incorporated.

In step S10, cleaning plan generator 160 executes cleaning in the normal mode based on the cleaning plan corresponding to the normal mode.

In step S11, map information corrector 150 reflects and corrects, in the map information stored in map storage unit 80, harmful concentrated region D1 determined by determination unit 110 during the execution of cleaning in the normal mode. Specifically, in step S11, processing similar to the processing in step S6 is performed.

In step S12, cleaning plan generator 160 creates a cleaning plan in the countermeasure mode.

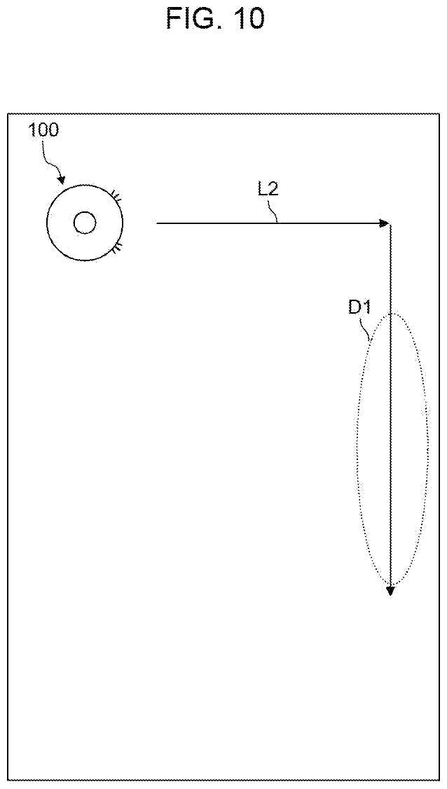

FIG. 10 is an explanatory diagram illustrating an example of a cleaning plan in the countermeasure mode according to the exemplary embodiment. As illustrated in FIG. 10, in the countermeasure mode, travel route L2 passing harmful concentrated region D1 is generated.

Returning to FIG. 6, in step S13, cleaning plan generator 160 executes cleaning in the countermeasure mode based on the cleaning plan corresponding to the countermeasure mode. At this time, autonomous cleaner 100 sprays the countermeasure agent from countermeasure agent spraying unit 37 to harmful concentrated region D1 while traveling along travel route L2 illustrated in FIG. 10. As a result, a countermeasure against a harmful substance is taken for harmful concentrated region D1.

When the countermeasure against harmful concentrated region D1 is completed, cleaning plan generator 160 outputs completion information to map information corrector 150. Map information corrector 150 updates the map information based on the completion information. Specifically, map information corrector 150 corrects the map information so that a display mode of harmful concentrated region D1 is different from the display mode before cleaning in the countermeasure mode, and causes input unit 70 to display the map information.

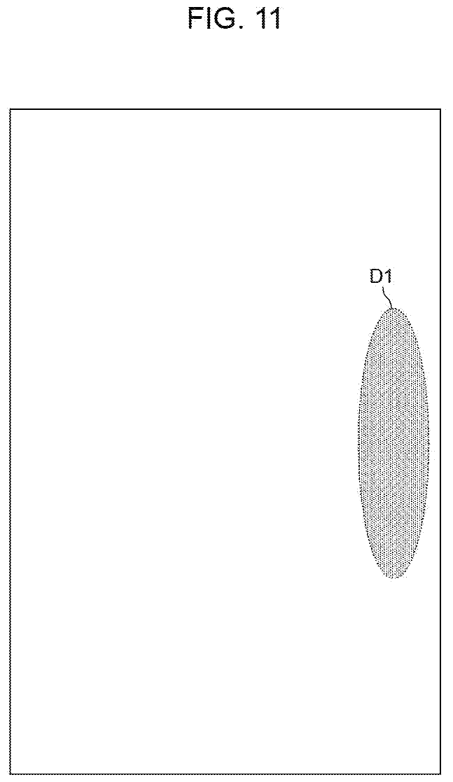

FIG. 11 is an explanatory diagram illustrating an example of a map displayed on input unit 70 according to the exemplary embodiment after completion of cleaning in the countermeasure mode. In FIG. 11, harmful concentrated region D1 is displayed in a color different from the color before cleaning. As a result, the user can visually recognize whether or not a countermeasure has been taken against harmful concentrated region D1.

### [Effects and Others]

As described above, autonomous cleaner 100 according to the exemplary embodiment includes: main body 10 including housing 11, drive wheels (wheels 20) attached to housing 11, and drive unit 25 that drives the drive wheels; map storage unit 80 that stores map information for main body 10 to travel; determination unit 110 that determines whether or not main body 10 approaches or comes into contact with a person infected with at least one of a virus and a bacterium; and map information corrector 150 that reflects, in the map information stored in map storage unit 80, contact information on approach or contact of main body 10 with the infected person determined by determination unit 110.

According to this configuration, since the contact information on approach or contact of main body 10 with the infected person is reflected and corrected in the map information, it is possible to specify the region (harmful concentrated region D1) where the infected person has been present in the predetermined space by confirming the corrected map information. Therefore, it is possible to take a countermeasure against the infected person.

In addition, it is possible to take an appropriate countermeasure only against harmful concentrated region D1 without taking a countermeasure against the entire predetermined space. This makes it possible to reduce the time required for a countermeasure against a virus, a bacterium, and the like.

In addition, the contact information includes at least either of information on a place where main body 10 approaches or comes into contact with the infected person or information on date and time.

According to this configuration, since the contact information includes at least either of the information on the place where main body 10 approaches or comes into contact with the infected person or the information on the date and time, it is possible to easily specify the place where the infected person has been present, and the date and time, and it is possible to take a countermeasure more easily.

Further, autonomous cleaner 100 includes communication unit 60 that communicates with communication terminal 250, communication unit 60 receives the infected person information of the user of communication terminal 250 from communication terminal 250, and determination unit 110 makes a determination based on the infected person information received by communication unit 60.

According to this configuration, it is determined whether or not main body 10 approaches or comes into contact with the infected person based on the infected person information received from external communication terminal 250. Therefore, it is possible to grasp whether or not the user of communication terminal 250 is an infected person without diagnosis by autonomous cleaner 100.

Further, autonomous cleaner 100 includes a display unit (input unit 70) that displays map information in which the contact information is reflected by map information corrector 150.

According to this configuration, since the map information in which the contact information is reflected is displayed on the display unit, an operator can confirm the corrected map on the spot.

Further, autonomous cleaner 100 includes controller 170 that controls drive unit 25 based on the map information, and a countermeasure executer (cleaning unit 35) that executes a countermeasure including at least one of reduction and prevention of a harmful substance including at least one of a virus and a bacterium. Controller 170 controls drive unit 25 and the countermeasure executer based on the map information, thereby directing main body 10 to a region (harmful concentrated region D1) where main body 10 approaches or comes into contact with the infected person to execute the countermeasure.

According to this configuration, since main body 10 itself moves to harmful concentrated region D1 and executes the countermeasure, it is possible to quickly execute the countermeasure against harmful concentrated region D1. It is also possible to reduce a risk of infection to cleaning personnel.

Note that the present disclosure may be realized as a program for causing a computer to execute steps included in a method for controlling autonomous cleaner 100. In this case, the method for controlling autonomous cleaner 100 according to the present exemplary embodiment can be easily executed by a computer.

In addition, the present disclosure may be realized as a non-transitory recording medium such as a compact disc read only memory (CD-ROM) readable by a computer in which the program is recorded. In addition, the present disclosure may be realized as information, data, or a signal indicating the program. Such a program, information, data, and signal may be distributed via a communication network such as the Internet.

### Other Exemplary Embodiments

The autonomous cleaner and the like according to the present disclosure have been described above based on the exemplary embodiment and the modification, but the present disclosure is not limited to the exemplary embodiment and the modification.

For example, in the above exemplary embodiment, a case has been exemplified where the display mode in harmful concentrated region D1 in input unit 70 (display unit) is changed before and after the countermeasure. However, the display unit may change the display mode of harmful concentrated region D1 as the time elapses after main body 10 approaches or comes into contact with the infected person. As a result, the operator can easily visually recognize passage of time after main body 10 approaches or comes into contact with the infected person. In general, a bacterium, a virus, or the like is inactivated over time. If the passage of time can be easily visually recognized, a degree of inactivation of a bacterium or a virus that may exist in harmful concentrated region D1 can also be estimated, and it is easy to take a countermeasure corresponding to the moment.

In addition, the display unit may display the color in the display mode to be lighter as the time elapses. In this case, the operator can intuitively estimate the degree of inactivation of a bacterium or a virus that may exist in harmful concentrated region D1 by shades of color.

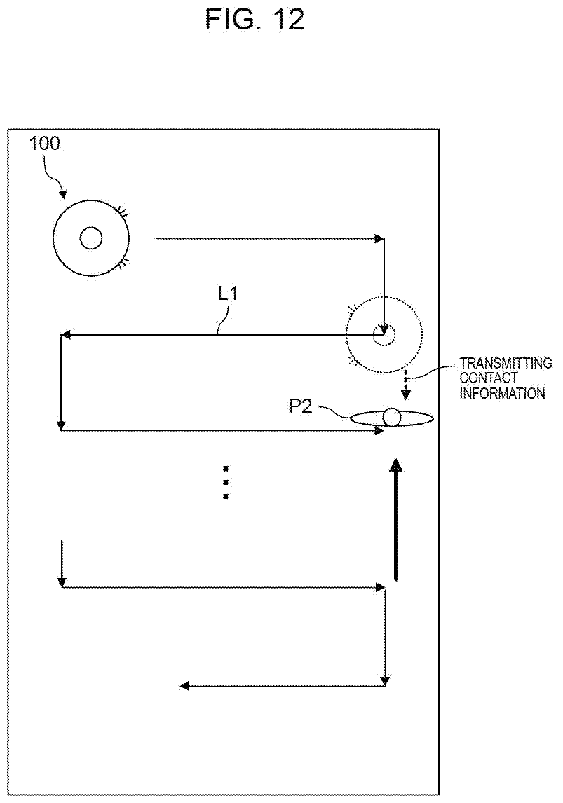

Further, in the above exemplary embodiment, a case has been exemplified where autonomous cleaner 100 acquires the infected person information from external communication terminal 250. However, autonomous cleaner 100 may transmit the contact information to external communication terminal 250. For example, even after the contact information is created, autonomous cleaner 100 performs cleaning, but also approaches or comes into contact with a person. Regardless of whether the person himself/herself is an infected person or a non-infected person, the person cares about the presence of an infected person in the past in the place where the person himself/herself is currently present. Therefore, after the contact information is created, communication unit 60 of autonomous cleaner 100 transmits the contact information to communication terminal 250 when communication unit 60 and another communication terminal 250 enter the mutual communication range. FIG. 12 is an explanatory diagram illustrating a case where autonomous cleaner 100 according to a modification transmits the contact information to communication terminal 250 of another person P2. As a result, since the contact information can be transmitted to communication terminal 250 of person P2 who is different from person P1, who is the basis of the creation of the contact information, it can also be recognized that person P2 has approached harmful concentrated region D1, and thus a countermeasure can be taken.

Further, in the exemplary embodiment described above, a case has been exemplified where autonomous cleaner 100 includes determination unit 110. However, the autonomous cleaner may not include the determination unit. That is, an entire cleaning system may have a function of correcting the map information.

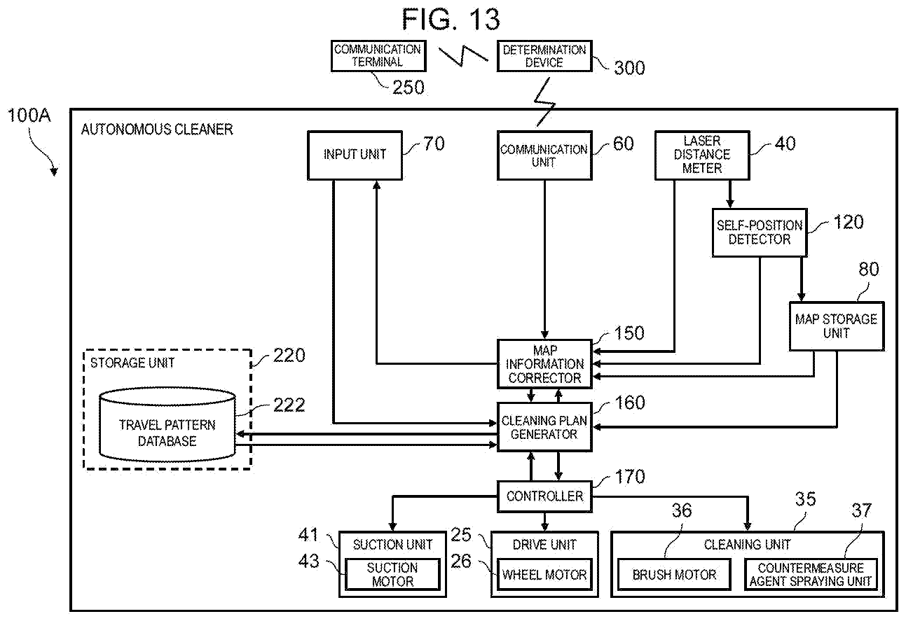

FIG. 13 is a block diagram illustrating a characteristic functional configuration of cleaning system 200 according to a modification. Specifically, FIG. 13 corresponds to FIG. 4. In the following description, the same parts as the parts in the above exemplary embodiment are denoted by the same reference numerals, and the description thereof may be omitted.

As illustrated in FIG. 13, cleaning system 200 includes autonomous cleaner 100A and determination device 300 that can freely communicate with autonomous cleaner 100A.

Determination device 300 is a communication terminal such as a smartphone or a tablet terminal. Determination device 300 is mounted on autonomous cleaner 100A, and also functions as an input unit and a display unit. Determination device 300 is communicably connected to communication unit 60 of autonomous cleaner 100A. Determination device 300 acquires the infected person information by communicating with external communication terminal 250, and determines whether or not determination device 300 approaches or comes into contact with an infected person. Determination processing is similar to the processing of determination unit 110 described above, and determination device 300 creates, as determination time information, a history of the time when determination device 300 determines that determination device 300 approaches or comes into contact with an infected person, and outputs the determination time information to communication unit 60.

In autonomous cleaner 100A, when the determination time information is input to map information corrector 150 through communication unit 60, map information corrector 150 creates the contact information based on the determination time information, and reflects and corrects the contact information in the map information.

As described above, according to cleaning system 200 of the modification, since the contact information including a determination result of determination device 300 is reflected and corrected in the map information, it is possible to specify the region where an infected person has been present in the predetermined space by confirming the corrected map information. Therefore, it is possible to take a countermeasure against the infected person.

Further, in the above exemplary embodiment, a case has been exemplified where autonomous cleaner 100 itself takes a countermeasure against harmful concentrated region D1. However, the operator may confirm the corrected map information and the operator may take a countermeasure against harmful concentrated region D1. Alternatively, the corrected map information may be read by another countermeasure device, and a countermeasure against harmful concentrated region D1 may be taken by the countermeasure device.

In the above exemplary embodiment, it has been described that processors such as a cleaning plan generator and a controller included in the autonomous cleaner are implemented by a CPU and a control program, respectively. For example, each of the components of the processors may include one or a plurality of electronic circuits. Each of the one or plurality of electronic circuits may be a general-purpose circuit or a dedicated circuit. The one or plurality of electronic circuits may include, for example, a semiconductor device, an integrated circuit (IC), a large scale integration (LSI), or the like. The IC or the LSI may be integrated on one chip or may be integrated on a plurality of chips. Although referred to as an IC or an LSI here, the terms vary depending on the degree of integration, and may be referred to as a system LSI, a very large scale integration (VLSI), or an ultra large scale integration (ULSI). A field programmable gate array (FPGA) programmed after manufacture of the LSI can also be used for the same purpose.

In addition, general or specific aspects of the present disclosure may be implemented by a system, a device, a method, an integrated circuit, or a computer program. Alternatively, the aspects may be realized by a computer-readable non-transitory recording medium such as an optical disk, a hard disk drive (HDD), or a semiconductor memory in which the computer program is stored. Alternatively, the aspects may be implemented with any combination of the system, the device, the method, the integrated circuit, the computer program, and the recording medium.

In addition, the present disclosure also includes embodiments obtained by applying various modifications conceived by those skilled in the art to the exemplary embodiments and the modifications, and embodiments realized by arbitrarily combining components and functions in the exemplary embodiments without departing from the gist of the present disclosure.

The present disclosure is widely applicable to an autonomous cleaner that performs cleaning while autonomously moving.

What is claimed is:

1. An autonomous cleaner comprising:
a main body including a housing, a drive wheel attached to the housing, and a drive unit that drives the drive wheel;
a map storage unit that stores map information for the main body to travel;
a determination unit that makes a determination of whether or not the main body approaches or comes into contact with a person infected with at least one of a virus and a bacterium; and
a map information corrector that reflects, in the map information stored in the map storage unit, contact information on approach or contact of the main body with the infected person determined by the determination unit.

2. The autonomous cleaner according to claim 1, wherein
the contact information includes at least either of information on a place where the main body approaches or comes into contact with the infected person or information on date and time.

3. The autonomous cleaner according to claim 1, further comprising
a communication unit that communicates with a communication terminal,
wherein
the communication unit receives infected person information of a user of the communication terminal from the communication terminal, and
the determination unit makes the determination based on the infected person information received by the communication unit.

4. The autonomous cleaner according to claim 1, further comprising a display unit that displays the map information including the contact information reflected by the map information corrector.

5. The autonomous cleaner according to claim 1, further comprising a display unit that displays the map information including the contact information reflected by the map information corrector, wherein
the display unit displays a place where the main body approaches or comes into contact with the infected person in a display mode that allows a user to identify the place, and changes the display mode as time elapses after the main body approaches or comes into contact with the infected person.

6. The autonomous cleaner according to claim 1, further comprising a display unit that displays the map information including the contact information reflected by the map information corrector, wherein
the display unit displays a color in the display mode to be lighter as the time elapses.

7. The autonomous cleaner according to claim 1, further comprising:
a controller that controls the drive unit based on the map information; and
a countermeasure executer that executes a countermeasure including at least one of reduction and prevention of a harmful substance including at least one of a virus and a bacterium,
wherein
the controller controls the drive unit and the countermeasure executer based on the map information to cause the main body to move toward a region where the main body approaches or comes into contact with the infected person and execute the countermeasure.

8. The autonomous cleaner according to claim 1, further comprising
a communication unit that communicates with a communication terminal,
wherein
the communication unit transmits the contact information to the communication terminal.

9. A cleaning system comprising:
an autonomous cleaner; and
a determination device configured to freely communicate with the autonomous cleaner,
wherein
the autonomous cleaner includes:
a main body including a housing, a drive wheel attached to the housing, and a drive unit that drives the drive wheel;
a map storage unit that stores map information for the main body to travel;
a communication unit that communicates with the determination device; and
a map information corrector that corrects the map information stored in the map storage unit,
wherein
the determination device determines whether or not the determination device approaches or comes into contact with a person infected with at least one of a virus and a bacterium, and
the map information corrector reflects, in the map information stored in the map storage unit, contact information on approach or contact of the determination device with the infected person determined by the determination device.
